(19) United States
(12) Patent Application Publication
(10) Pub. No.: US 2022/0370597 A1
(43) Pub. Date: Nov. 24, 2022

(54) RECOMBINANT PROTEIN AND VACCINE COMPOSITION OF PORCINE EPIDEMIC DIARRHEA VIRUS

(71) Applicant: Jiangsu Academy of Agricultural Sciences, Nanjing (CN)

(72) Inventors: Bin Li, Nanjing (CN); Baochao Fan, Nanjing (CN); Danyi Shi, Boli County (CN); Jinzhu Zhou, Nanjing (CN); Rongli Guo, Nanjing (CN); Kongwang He, Nanjing (CN); Yongxiang Zhao, Nanjing (CN); Xuejiao Zhu, Nanjing (CN); Li Li, Nanjing (CN); Wei Wang, Nanjing (CN); Dandan Wang, Nanjing (CN); Haodan Zhu, Nanjing (CN)

(21) Appl. No.: 17/586,211

(22) Filed: Jan. 27, 2022

(30) Foreign Application Priority Data
May 19, 2021 (CN) 2021105465363

(51) Int. Cl. A61K 39/215; C07K 14/005

(52) U.S. Cl. CPC A61K 39/215; C07K 14/005; C07K 2319/31; C12N 2770/20022; C12N 2770/20031; A61K 2039/55505

(57) ABSTRACT

A recombinant protein and a vaccine composition for porcine epidemic diarrhea (PED) are provided. The recombinant protein is a fusion protein formed by connecting the truncated segment of S protein (Spike protein) from porcine epidemic diarrhea virus (PEDV) in tandem with the Fc fragment of porcine IgG, and the truncated fragment of S protein is preferably selected from N-terminal domain (NTD) with sialic acid binding activity in S1 subunit of S protein, neutralizing epitope domain (COE) and multiple B-cell epitopes in S2 subunit; the vaccine composition contains recombinant protein and adjuvants. The recombinant protein of the application can produce IgG antibody and neutralizing antibody titers of rather high level after immunizing mice, and the proportions of CD3⁺CD4⁺, CD3⁺CD8⁺ lymphocytes and the concentrations of IFN-γ and IL-4 in lymphocytes are significantly increased.

### TECHNICAL FIELD

The disclosure belongs to the field of biotechnology, and particularly relates to a recombinant protein a vaccine composition of porcine epidemic diarrhea virus.

### BACKGROUND

Porcine epidemic diarrhea (PED) is a highly infectious intestinal disease caused by PED virus (PEDV), a Class I coronavirus; the diseased animal has symptoms including severe watery diarrhea, vomiting and dehydration and some systemic symptoms such as vomiting, fever, anorexia and lethargy; piglets, in particular, are more vulnerable to the virus and their symptoms of dehydration are more severe. Until now, the virus has caused huge economic losses to the worldwide swine industry.

At present, the market available vaccines for PEDV are conventional inactivated PEDV vaccines and attenuated vaccines; however, inactivated vaccines are not suitable for emergency vaccination as a single time of vaccination of the vaccine cannot produce enough antibody, which justify a large dose of vaccination as to realize desired efficacies; besides, there are risks of virus spreading and retest positive among pigs vaccinated with attenuated vaccine.

### SUMMARY

In view of the problems existing in the prior art, a recombinant protein is provided by the disclosure on one hand, wherein the recombinant protein is a fusion protein formed by connecting a truncated fragment/segment of S protein (Spike glycoprotein) from PEDV and the Fc segment of porcine IgG in series.

The term PEDV S protein refers to one of the main structural proteins of PEDV consisting of 1,383 amino acids. It is one of the main envelope glycoprotein (Env) responsible for attachment, receptor binding and entry of the virus. According to its homology with other coronavirus S proteins, S protein of PEDV can also be classified as 51 subunit (1-725 aa), mediating the attachment of virus and cell surface receptor; and S2 subunit (726-1,383 aa), participating in the fusion of viruses and host cell membrane.

The term Fc fragment of porcine IgG refers to CH2 and CH3 of the constant region of porcine immunoglobulin G heavy chain, including natural amino acid sequence and its sequence derivatives (i.e. mutants).

The truncated segment of S protein in the recombinant protein of the disclosure is preferably selected from N-terminal domain (NTD) with sialic acid binding activity in 51 subunit of S protein, neutralizing epitope domain (COE) and multiple B-cell epitopes in S2 subunit.

Further, preferably, the truncated segment of the S protein comprises: a segment with the amino acid sequence shown in SEQ ID NO:1 or an amino acid sequence with same function as and formed by substituting, deleting and/or adding with one or more amino acid residues into the amino acid sequence as shown in SEQ ID NO:1, a segment with the amino acid sequence shown in SEQ ID NO:2 or an amino acid sequence having the same function as and formed by substituting, deleting and/or adding with one or more amino acid residues into the amino acid sequence as shown in SEQ ID NO:2; and a segment with the amino acid sequence shown in SEQ ID NO:3 or an amino acid sequence having the same function as and formed by substituting, deleting and/or adding with one or more amino acid residues into the amino acid sequence as shown in SEQ ID NO:3.

The Fc fragment of porcine IgG preferably has the amino acid sequence as shown in SEQ ID NO:4.

Preferably, the recombinant protein contains or comprises the amino acid sequence as shown in SEQ ID NO:5 or an amino acid sequence with the same function as and formed by substituting, deleting and/or adding with one or more amino acid residues into the amino acid sequence as shown in SEQ ID NO:5.

Preferably, the recombinant protein is a recombinant protein obtained by exogenously expressing a recombinant gene with the deoxyribonucleic acid (DNA) sequence as shown in SEQ ID NO:6 by using an eukaryotic expression system.

Preferably, the recombinant protein is a recombinant protein obtained by exogenously expressing a recombinant gene with the DNA sequence as shown in SEQ ID NO:6 by using a mammalian cell expression system.

The disclosure also provides an application of the recombinant protein for preparing PEDV vaccine and/or detecting PEDV antibody.

A vaccine composition of PEDV is provided on another hand by the disclosure, and the composition contains the recombinant protein of the disclosure and a pharmaceutically acceptable adjuvant.

Preferably, the adjuvant is Freund's adjuvant or a layered double hydroxide (LDH) adjuvant.

When adopting the Freund's adjuvant as adjuvant, the vaccine composition is prepared by mixing and emulsifying the recombinant protein with the adjuvant in a volume ratio of 1:1. When using LDH as the adjuvant, the vaccine composition is prepared by mixing and emulsifying the recombinant protein with the adjuvant in a mass ratio of 1:4.

Preferably, the LDH adjuvant is prepared by the following method: 51, preparing a first mixed solution containing a mixed salt solution of 0.54 mol/L Mg(NO₃)₂ and 0.27 mol/L Al(NO₃)₃; S2, preparing a second mixed solution, wherein the second mixed solution is prepared by mixing NaOH and lactic acid and vigorously stirring; S3, mixing the first mixed solution and the second mixed solution to react, after which ultrasonically treating the precipitate in an ice bath for 10 min (minute), performing centrifuging at 5,000 rpm for 10 min to obtain pure LDH slurry, washing the slurry with water twice, and then dispersing it in 20 mL of water to obtain the LDH adjuvant in the form of nanoparticles.

The vaccine composition of the disclosure is effective for preventing and treating PED.

According to the disclosure, genes expressing functional epitopes (NTD, COE and B cell epitopes) of a plurality of truncated segments of PEDV S protein are concatenated in series, and genes expressing porcine IgG Fc segments are further concatenated in series, so that an eukaryotic expression plasmid is constructed to efficiently express recombinant proteins through an eukaryotic expression system, the purification process of the recombinant proteins expressed by the eukaryotic expression system is simple and convenient with high yields, and large-scale production is therefore facilitated.

The disclosure further utilizes the recombinant protein obtained by exogenous expression to prepare subunit vaccine, and the prepared vaccine has advantages of high safety, good immunogenicity, batch-to-batch stability in addition to low producing costs, capability of inducing pigs to generate good immune response and the like.

### BRIEF DESCRIPTION OF DRAWINGS

FIG. 1 illustrates the plasmid profile of pcDNA3.1-S1-Fc.

FIG. 2 shows the results of expressing recombinant eukaryotic-expressed plasmid pcDNA3.1-S1-Fc in 293T cells by indirect immunofluorescence.

FIG. 3 shows the results of Western blot detection of the eukaryotic-expressed recombinant protein, in which M is the molecular quality standard of protein, and lanes 1 and 2 are pcDNA3.1-PEDV eukaryotic proteins.

FIG. 4 shows the results of detecting the particle size of the prepared LDH adjuvant using Nicomp 380 Z3000 nanometer particle size analyzer.

FIG. 5 shows the results of detecting IgG antibody in serum of mice immunized with the recombinant protein of the embodiment of the disclosure.

FIG. 6 shows the results of detecting neutralizing antibody titer in mouse serum after vaccine immunization.

FIGS. 7A-7D and 8 illustrate the results of detecting the proportion of CD3+CD4+subtypes of mouse spleen lymphocytes by flow cytometry, in which FIG. 7A shows a blank group, FIG. 7B represents vaccine composition 1, FIG. 7C represents vaccine composition 2, and FIG. 7D represents PBS control.

FIGS. 9A-9D and 10 illustrate the results of detecting the proportion of CD3+CD8+subtypes of mouse spleen lymphocytes by flow cytometry, in which FIG. 9A shows a blank group, FIG. 9B represents vaccine composition 1, FIG. 9C represents vaccine composition 2, and FIG. 9D represents PBS control.

FIG. 11 shows the results of detecting the relative proliferation rate of mouse spleen lymphocytes by MTT method.

FIGS. 12 and 13 illustrate the detection results of mouse cytokines IFN-γ (interferon-γ). and IL-4 (interleukin-4).

### DETAILED DESCRIPTION OF EMBODIMENTS

The technical scheme of the disclosure will be further described below with reference to the drawings and specific embodiments, and the advantages and characteristics of the disclosure will become clearer with the description. However, it should be understood that the embodiments are only exemplary and do not limit the scope of the disclosure. Those skilled in the art should understand that the details and forms of the technical scheme of the disclosure can be modified or replaced without departing from the spirit and scope of the disclosure, but these modifications and substitutions fall within the scope of protection of the disclosure.

In the following description, unless otherwise specified, the methods involved are all conventional methods in this field, and the raw materials involved, unless otherwise specified, are all available from open commercial channels.

### Embodiment 1: Construction of Recombinant Plasmid for Eukaryotic

Expression of Recombinant Protein

In this embodiment, a fusion gene with the DNA sequence shown in SEQ ID NO:6 is synthesized by Genscript Biotech Corporation in Nanjing based on the S gene sequence of PEDV AH2012/12 strain (GenBank:KU646831.1) and is named as S1-Fc; the fusion gene S1-Fc contains three truncated segments of S protein gene, namely the N-terminal domain (NTD, aa19-233) with sialic acid binding activity in 51 subunit, the core neutralizing epitope (COE, aa499-638) and the B-cell epitope (aa747-774) in S2 subunit, and these three truncated fragments are sequentially linked by a linker peptide, followed by the fusion of the Fc fragment of the porcine IgG antibody.

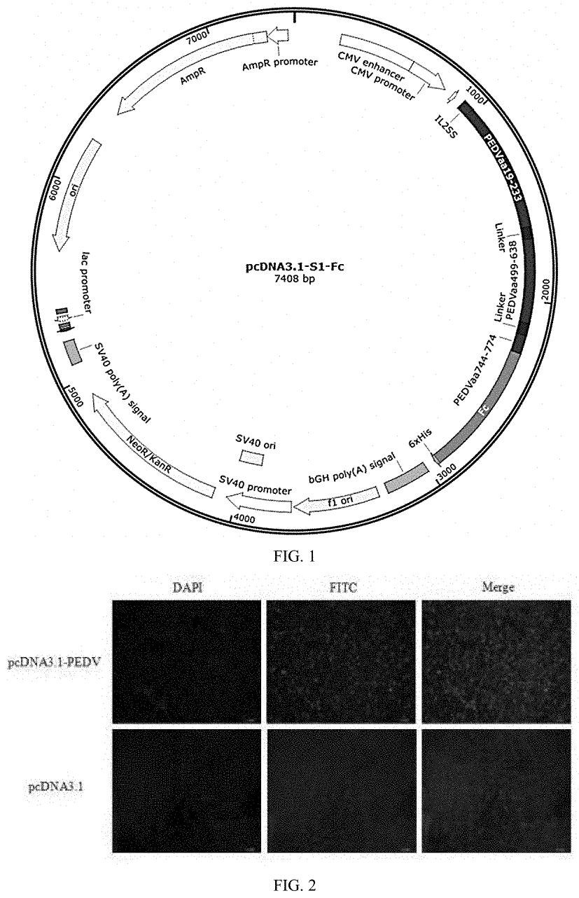

The synthesized fusion gene is cloned onto plasmid vector pcDNA3.1 by conventional molecular biological methods, the recombinant plasmid pcDNA3.1-S1-Fc for expressing recombinant protein is hence constructed, and its diagram is as shown in FIG. 1.

Competent cells of Escherichia coli DH5a is transformed by the recombinant plasmid pcDNA3.1-S1-Fc to construct the recombinant Escherichia coli for amplifying the recombinant plasmid pcDNA3.1-S1-Fc, then the recombinant Escherichia coli is inoculated onto LB liquid medium containing 100 ng/mL ampicillin and cultured at 37° C. and shaking of 200 rpm for 8 h (hour) in an incubator under constant temperature, and the recombinant plasmid pcDNA3.1-S1-Fc is extracted and purified from the culture with the plasmid extraction kit from Axygen Company.

The purified recombinant plasmid pcDNA3.1-S1-Fc is transfected by transfection reagent Lipofectamine3000 (purchased from Invitrogen Company) into 293T cell, meanwhile, the plasmid vector pcDNA3.1 is transfected as negative control, and the expression of the fusion gene S1-Fc is detected by indirect immunofluorescence.

When the 293T cells are passaged and paved, a cell slide is put into the culture well, and the 293T cells are transfected with the recombinant plasmid pcDNA3.1-S1-Fc and then cultured; when specific lesions of cells are observed (i.e., shrinkage of cells and slight cellular lesions can be observed), the cells are washed with PBS for three times, and absolute ethanol is added in an amount of 500 μL/well to fix at 4° C. for 15 min; after the fixation, washing with PBST is performed for three times and a primary antibody (mouse anti-HIS-tagged monoclonal antibody, purchased from Genscript Company, USA, diluted 1,000 times) is added, and then it is incubated in a constant temperature incubator for 30 min; washing with PBST is then performed twice before adding secondary antibody (FITC goat anti-mouse IgG antibody, Boster Biological Technology Co., Ltd., diluted 500 times); after the twice washing with PBST, DAPI dye solution (diluted 1,500 times) is added to react for 5 min; then the cell slide is moved out and fixed on a glass slide, and the fluorescence is observed with an inverted fluorescence microscope.

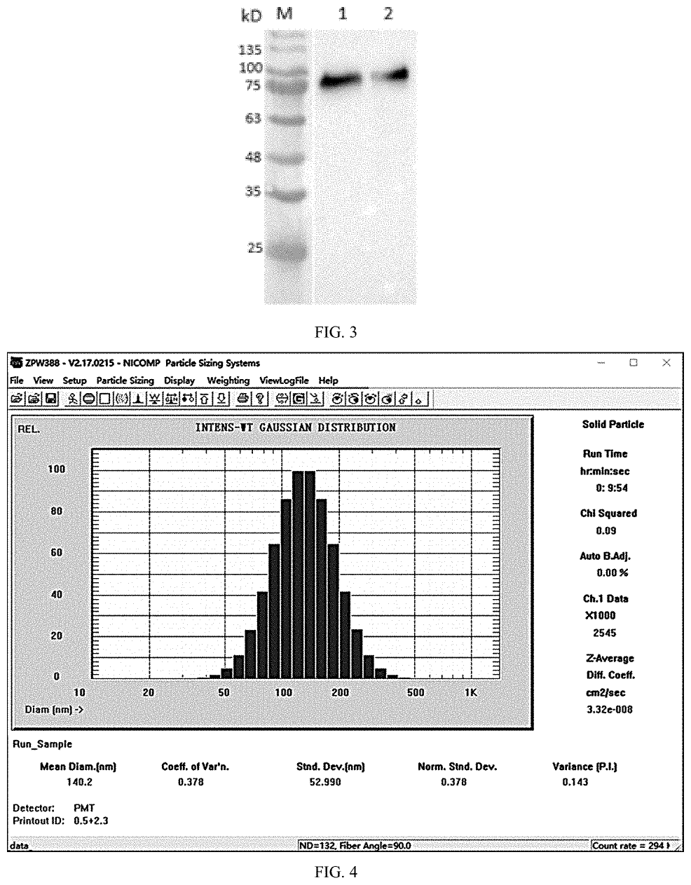

As shown in FIG. 2, the culture wells transfected with recombinant plasmid pcDNA3.1-S1-Fc show obvious specific fluorescence reaction, while the control wells transfected with plasmid vector pcDNA3.1 show no fluorescence reaction, indicating that the recombinant plasmid pcDNA3.1-S1-Fc constructed above can successfully drive the expression of the target gene in 293T cells.

### Embodiment 2: Preparation of Eukaryotic Expression Recombinant Protein

In this embodiment, a large number of recombinant proteins are prepared by Expi293™ expression system. The ExpiFectamine™293 transfection kit used in this embodiment is purchased from Gibco Company.

The specific operation is as follows:

(1) inoculating 6*10⁷ Expi293F™ living cells in 30 mL Expi293™ expression medium;

(2) incubating cells in an orbital shaker of 125 rpm at 37° C. with 8% CO2;

(3) determining the number and vitality of cells with an automatic cell counter, the cell density should be 3*10⁶-5*10⁶ cells/mL, and the cell vitality should be greater than 95%;

(4) diluting 7.5*10⁷ cells to 2.9*10⁶ cells/mL in a 125 mL flask with 25.5 mL Expi293™ expression medium;

(5) diluting 30 ng pcDNA3.1-S1-Fc eukaryotic expression plasmid with Opti-MEM in tube A to a total volume of 1.5 mL, diluting 81 μL ExpiFectamine™293 reagent with Opti-MEM in tube B to a total volume of 1.5 mL, gently mixing the tubes respectively and then incubating at room temperature for 5 min, then adding the mixture in tube B to tube A to obtain a compound with a total volume of 3 mL, and gently mixing well and incubating under room temperature for 20 min;

(6) adding 3 mL of the above compound into the flask, at which time the volume of the mixture in the flask is 28.5 mL;

(7) incubating the cells in an orbital shaker at 125 rpm and 37° C. with 8% CO2 for 20 h;

(8) adding 150 μL of ExminFectamin™293 transfection enhancer 1 and 1.5 mL of Exponentamine™293 transfection enhancer 2 into the flask, the volume of the mixture in the flask at the end should be about 30 mL;

(9) collecting protein.

The expression of recombinant protein is detected by Western blot.

20 μL of protein samples is taking out from the collected proteins and added with 5 μL of 5*SDS-PAGE loading buffer, boiled for 10 min, and then the SDS-PAGE gel electrophoresis is performed.

After SDS-PAGE electrophoresis, the protein on the gel is transblotted to nitrocellulose membrane, and then the nitrocellulose membrane is immersed in PBST containing 5% skim milk and sealed for 2 h. After blocking, the membrane is washed with PBST for three times, then the mouse anti-HIS-tagged monoclonal antibody (purchased from Genscript Company, USA, diluted by 2,000 times) is added as the primary antibody, slow-shaking is performed for 90 min; washing with PBST is performed for three times before HRP enzyme-labeled goat anti-mouse IgG antibody (purchased from Boster Biological Technology Co., Ltd., diluted by 10,000 times) is added as secondary antibody, slow-shaking is performed for 60 min followed by washing with PBST for 3 times; regent I and regent II are mixed in equal amount to evenly soak the nitrocellulose membrane, and the results are observed by chemiluminescence instrument.

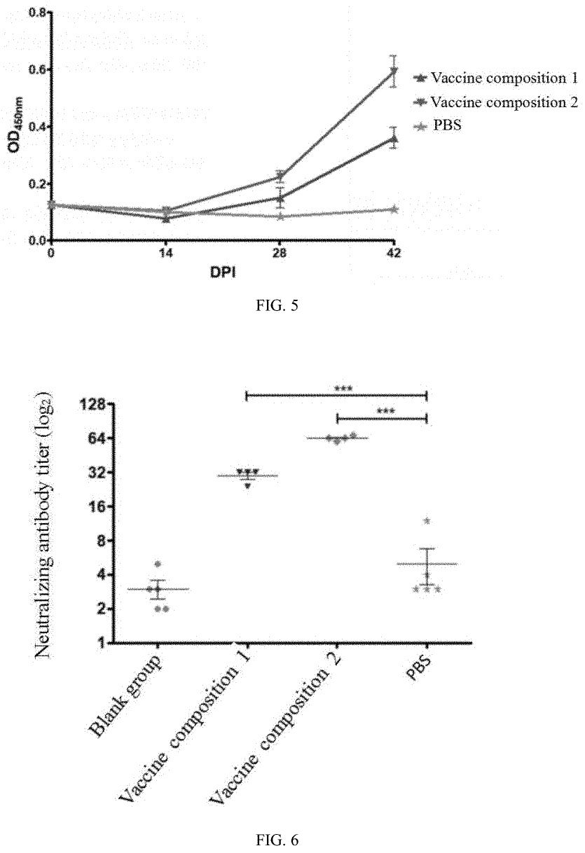

It can be seen from the results as shown in FIG. 3 that a large number of recombinant proteins can be successfully expressed using the ExpiFectamine™293 eukaryotic expression system.

Next, the protein obtained above is further purified by Protein G column, and the specific operation is as follows:

(1) adding 5 mL ddH₂O into a pre-loaded column to wash the storage solution in the chromatographic column;

(2) adding 5 mL binding buffer to the pre-loaded column to balance the chromatographic column;

(3) adding the sample protein into a well-balanced chromatographic column with a flow rate of 1 mL/min, and collecting the effluent;

(4) adding 10 mL of wash buffer to the pre-loaded column for cleaning, removing non-specifically adsorbed impurity proteins, and collecting the washed solution; and

(5) adding 10 mL elution buffer to the pre-loaded column, washing off the target protein combined with the chromatographic column, and collecting the eluent to obtain the purified recombinant protein.

The concentration of purified recombinant protein is 0.68 mg/mL detected by RC DC Protein Assay kit.

### Embodiment 3: Preparation of Vaccine Composition

Commercially available Freund's adjuvant and LDH adjuvant prepared by the inventor are mixed with the purified recombinant protein prepared in embodiment 2 to prepare a vaccine composition.

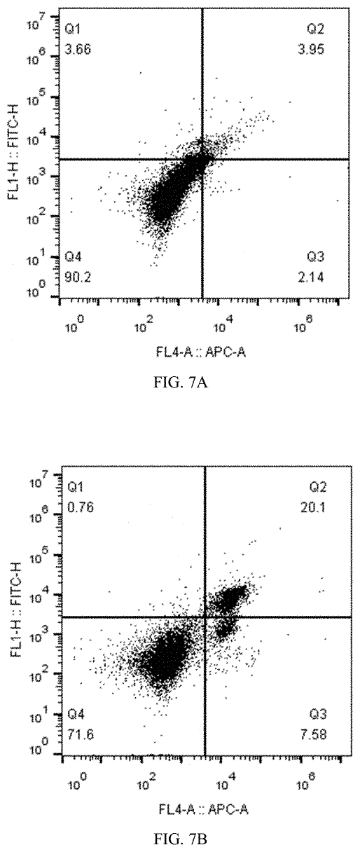

The preparation of LDH adjuvant is as follows: firstly, 15 mL of mixed salt solution containing 8.0 mmol of Mg(NO₃)₂ and 4.0 mmol of Al (NO₃)₃ is prepared; then, 20 mL of 4.0 mol/L NaOH solution is added into 20 mmol lactic acid (88%), and the mixture is stirred vigorously for 2 h to obtain NaOH mixture; then, the 15 mL of mixed salt solution is added into 11 ml of the NaOH mixture for reaction, and the precipitate generated after the reaction is ultrasonically treated in ice bath for 10 min, and centrifuged at 5,000 rpm for 10 min to obtain pure LDH slurry, which is washed twice with water and then manually dispersed in 20 mL of water to obtain prepared LDH adjuvant; the particle size of LDH adjuvant is measured by particle size analyzer Nicomp 380 Z3000 nanometer, and the results are as shown in FIG. 4, wherein the abscissa refers to the particle diameter, the ordinate represents the relative percentage of particles; it can be seen that a relatively strong facilitation effect may be achieved when the optimal size of LDH adjuvant is about 115 nm, and the results show that the size of LDH adjuvant made by the inventors is suitable.

The purified recombinant protein in embodiment 2 is mixed with LDH adjuvant in a mass ratio of 1:4 and emulsified to prepare vaccine composition 1, which is stored at 4° C. for later use. The purified recombinant protein in embodiment 2 is mixed with Freund's adjuvant in a volume ratio of 1:1 and emulsified to prepare vaccine composition 2, which is stored at 4° C. for later use.

### Embodiment 4: Evaluation of Immune Efficacy of Vaccine Compositions

20 BALB/c female mice aged 6-8 weeks (purchased from Experimental Animal Center of Yangzhou University) are randomly grouped into four groups, including two groups of vaccine group, a control group and a blank group, with five mice in each group. The immunization plan is shown in Table 1.

TABLE 1
Animal experimental immunization plan

| | | Immunogenic | | Immune | Quantity of | Immune |
|---|---|---|---|---|---|---|
| Group | Immunogen | dose | Adjuvant | dose | animals | route |
| Blank group | No operation | | | | | |
| Vaccine | Recombinant | 20 μg/ | LDH | 100 μL/ | 5 Mouse | Hypodermic |
| composition 1 | protein | Mouse | | Mouse | | injection |
| Vaccine | Eukaryotic | 20 μg/ | Freund's | 100 μL/ | 5 Mouse | |
| composition 2 | protein | Mouse | adjuvant | Mouse | | |
| PBS | PBS | — | — | 100 μL/ | 5 Mouse | |
| | | | | Mouse | | |

A total of three immunizations are conducted with an interval of 2 weeks. Blood samples are collected from orbital venous plexus 14th, 28^{th}and 42^{nd} days before and after immunization, and the serums are separated and stored at −20° C. to determine the serum antibody levels in different immunization periods.

The antibody titer is measured on the 42^{nd} day, wherein the blood samples are collected from eyeballs of every mouse, and the serums are separated, followed by measurement of antibody IgG and neutralization titer; spleen lymphocytes are isolated and cell measurement against MTT, CD3+CD4+ and CD3+CD8+ cells are performed; IL-4 and IFN-γ cytokines are measured in collected serum and spleen lymphocyte culture supernatants.

1. Determination of Serum IgG in Immunized Mice

The serums IgG level of immunized mice are detected by indirect Enzyme-linked immunosorbent assay (ELISA).

The prokaryotic recombinant COE protein with the amino acid sequence shown in SEQ ID NO:2 is used as antigen, and it is titrated with PEDV negative serum and positive serum to determine the optimal coating concentration of antigen and optimal dilution of serum; the recombinant COE protein is obtained by cloning the COE gene fragment with the sequence shown in SEQ ID NO:7 onto pGEX-4T-1 vector, and then expressing it in Escherichia coli BL21(DE3).

The purified prokaryotic recombinant COE protein is diluted with coating solution at 1:100, 1:200, 1:400, 1:800, 1:1600 and 1:3,200 in turn, which are then dropped into polystyrene microtiter plate at 100 μL/well followed by standing overnight at 4° C.; the next day, the liquid is discarded, and the well is washed with PBST 200 μL/well for three times, each time for 5 min; the well is added with 5% skimmed milk diluted by PBST at 200 μL/well and sealed at 37° C. for 2 h, then the well is washed again with PBST at 200 μL/well for three times, each time for 5 min; PEDV negative serum and positive serum are diluted at 1:50, 1:100, 1:200 and 1:400 with skim milk of 2%, respectively, to form a chessboard matrix, with 100 μL/well and 37° C. for 1 hour; then washing with PBST is performed for 3 times at 200 μL/well after discarding the liquid, each time for 5 min; goat anti-porcine IgG-HRP diluted with skim milk of 2% at 1:10,000 is added according to 100 μL/well at 37° C. for 1 h, then washing with PBST is performed for 3 times at 200 μL/well after discarding the liquid, each time for 5 min; TMB developing solution is added according to 100 μL/well and reacted away from light for 15 min; stop buffer is added according to 100 μL/well to stop the reaction, and the OD₄₅₀ₘₘ value is read with microplate reader; the positive serum with OD₄₅₀ₘₘ value about 1.0 and the negative serum with OD450mm value about 0.2 are selected, wherein the antigen coating concentration where the ratio of positive to negative OD₄₅₀ₘₘ (P/N) is maximized is the optimal antigen coating concentration, and the corresponding negative and positive serum dilutions are the optimal serum dilutions; at last, the optimal coating concentration of prokaryotic recombinant COE protein is determined to be 6.7 μg/mL, and the optimal dilution of serum is 1:50.

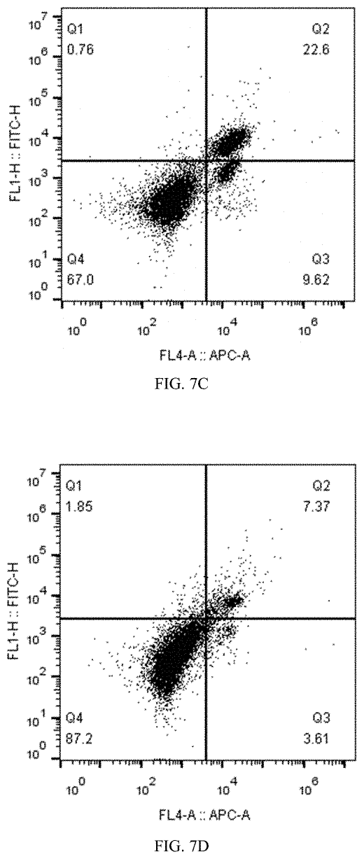

A polystyrene microtiter plate is coated under the best antigen coating concentration determined above, and IgG in serum collected on 14^{th}, 28^{th} and 42^{nd} days after immunization are detected by the above ELISA method; the results as shown in FIG. 5 indicate that the IgG antibody levels in the serum of mice in vaccine composition 1 and vaccine composition 2 groups are significantly higher than those in PBS group and blank group.

2. Neutralization Experiment of Immunized Mice Serum

(1) all the collected serums are subpackaged into a water bath and inactivated at 56° C. for 30 min;

(2) on the 96-well micro-cell culture plate, the inactivated serum is multiply diluted by pure DMEM at 1:2, 1:4, 1:8, 1:16, 1:32, 1:64, 1:128, 1:256 of the original serum, respectively; the final content of each well is 50 μL, and each dilution is repeated for 2 wells;

(3) the PEDV virus solution stored in the refrigerator at −80° C. is diluted by pure DMEM containing 5 μg/mL trypsin 200 times according to the measured TCID₅₀(mixed with the same amount of serum with toxicity of 100TCID₅₀);

(4) each well is added with 50 μL, of virus solution prepared in step (3) and covered, and neutralized in an incubator with 5% CO₂ at 37° C. for 1 h;

(5) when the 96-well plate is full of single layer VERO cells, the supernatant is discarded, and the plate is cleaned once with pure DMEM, then each well is added with mixture of serum and virus of 100 μL, to incubate in an incubator at 37° C. with 5% CO2 for 1.5 h; pure DMEM is used for gently washing the plate twice, then pure DMEM maintenance solution containing 2 μg/mL trypsin is used for replenishment; then it is put into the incubator for cultivation, and day-by-day observation and recording are started after 24 h.

The maximum dilution of the serum that completely inhibits the cytopathic effect (CPE) is used as the neutralizing titer of the serum; in order to ensure the reliability of the experimental results, a negative control with only virus solution and no serum in the well is arranged, wherein CPE must appear in the negative control well.

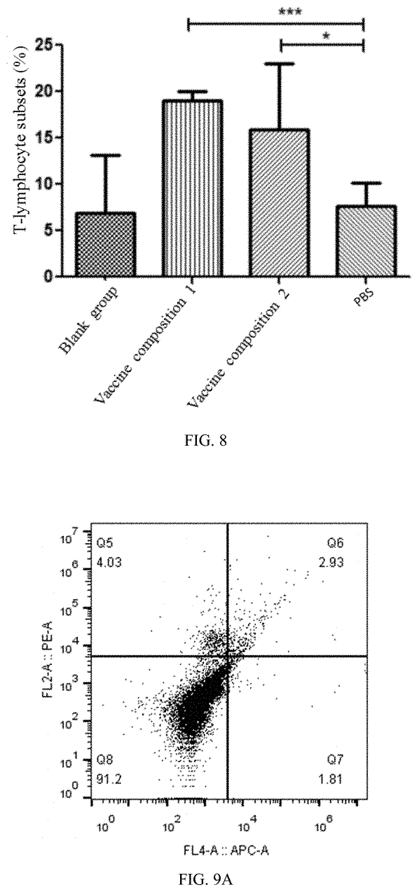

As shown in FIG. 6, the neutralizing antibody titer of vaccine composition 1 and vaccine composition 2 are 1:30 and 1:64, respectively, which are significantly higher than those of PBS group and blank group; especially, the vaccine combination 2 group has the highest neutralizing antibody titer and the most stable results in parallel repeats.

3. Flow Cytometry Detection of Splenic Lymphocyte Subtype Ratios

3.1 Preparation of Spleen Lymphocytes

The spleen of immunized mice is taken out and put into a plate containing RPMI-1640 culture medium (incomplete 1640) without bovine serum, and transferred to 300 mesh gauze, added with 10 mL of incomplete 1640 culture medium, gently ground with the top of the piston of sterile syringe, then added with incomplete 1640 culture medium to rinse the remaining tissue on the gauze into the plate; the plate is stood upright for 2 min, and the supernatant is sucked into a 15 mL sterile centrifuge tube after the tissue is precipitated, and the tube is centrifuged at 1,000 rpm for 10 min; then the supernatant is discarded, 5 mL sterile NH₄Cl (8.3 g/L) is added into the remaining material and mixed evenly; after standing at 37° C. for 5 min, the tube is again centrifuged at 1,000 rpm for 5 min; the precipitate is re-suspended with incomplete 1640 culture medium, followed by washing and centrifugation; and finally the cells are re-suspended with complete 1640 culture medium to obtain the prepared splenic lymphocytes.

3.2 Detection of the Proportion of Spleen Lymphocyte Subtypes

1*10⁶ mouse spleen lymphocytes prepared above are put into a 1.5 mL sterile centrifuge tube and centrifuged at 1,500 rpm for 5 min; after discarding the supernatant, the tube is washed with 1 mL PBS buffer and centrifuged again at 1,500 rpm for 5 min, the supernatant is discarded; then 300 μL PBS containing APC anti-mouse CD3, FITC rat anti-mouse CD4 (L3T4) and PE rat anti-mouse CD8a fluorescent antibody is used to re-suspend the cells, followed by completely mixing and incubating at 4° C. in the dark for 30 min; then the tube is washed with 1 mL PBS twice and centrifuged at 1,500 rpm for 5 min, and the supernatant is discarded; 500 μL flow cell staining buffer is used to re-suspend the cells at the bottom of the tube, and the proportions of CD3+CD4+ and CD3+CD8+positive cells in 10,000 cells are detected by BD Accuri™ C6 Plus flow cytometry.

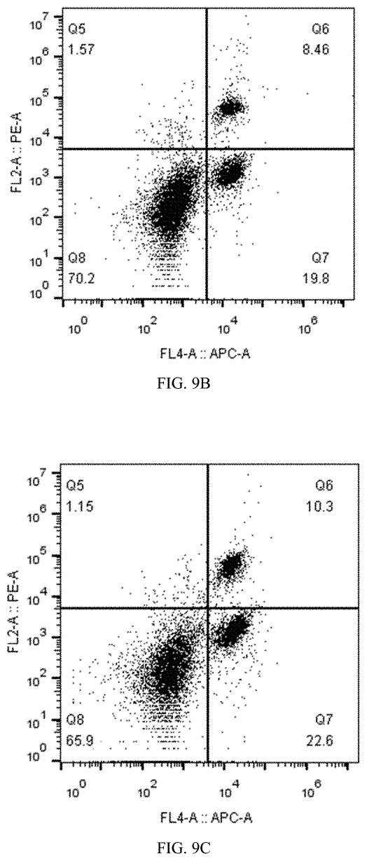

FIGS. 7A-7D and FIG. 8 show the changes of CD3+CD4+T lymphocytes. Compared with PBS group (7.64%) and blank group (6.88%), 42 days after the first immunization, the percentages of CD3+CD4±T lymphocytes in experimental groups of vaccine composition 1 and vaccine composition 2 increase to a certain extent, respectively, and the percentages in vaccine composition 1 and vaccine composition 2 groups are elevated by 11.43% and 8.26%, respectively.

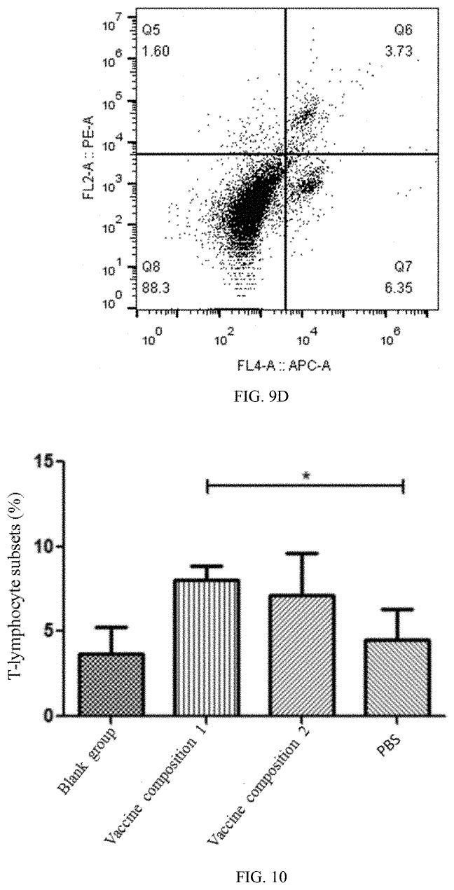

FIGS. 9A-9D and FIG. 10 show the changes of CD3+CD8±T lymphocytes. Compared with PBS group (4.52%) and blank group (3.66%), 42 days after the first immunization, the percentages of CD3⁺CD8⁺T lymphocytes in the experimental groups of vaccine composition 1 and vaccine composition 2 show a certain increase; specifically, the percentages of experimental groups of vaccine composition 1 and vaccine composition 2 go up by 3.48% and 2.61% respectively.

3.3 Detection of Mouse Spleen Lymphocyte Proliferation by MTT Method

Spleen lymphocyte single cell suspension is inoculated into 96-well plate with 10³-10⁴ cells/well with a volume of 200 uL/well, ConA with a final concentration of 10 μg/mL is added to each well after the cells adhere to the wall, RPMI-1640 culture medium with equal volume is added to the control well and 3 repeated wells are arranged; the cell plates are cultured in an incubator with 5% CO₂ at 37° C.; after 3-5 days of culture, 20 μL MTT solution (5 mg/mL, prepared with PBS) is added to each well, and then the culture is terminated after 4 h of culture; the culture supernatant in the well is carefully sucked out and preserved (3 wells of each sample are blended to 1 well with a totally amount of 300 μL for subsequent detection of cytokines); then, each well is added with 150 μL of DMSO and shaken for 10 min to fully melt the crystal; the light absorption value of each well is detected at the selected wavelength of 490 nm on the enzyme-linked immunosensor, and results are expressed as the average values of three repeated wells; finally, the relative cell proliferation rate (P %) is calculated as: P %=(average OD value of the experimental group/average OD value of the normal control group)*100%.

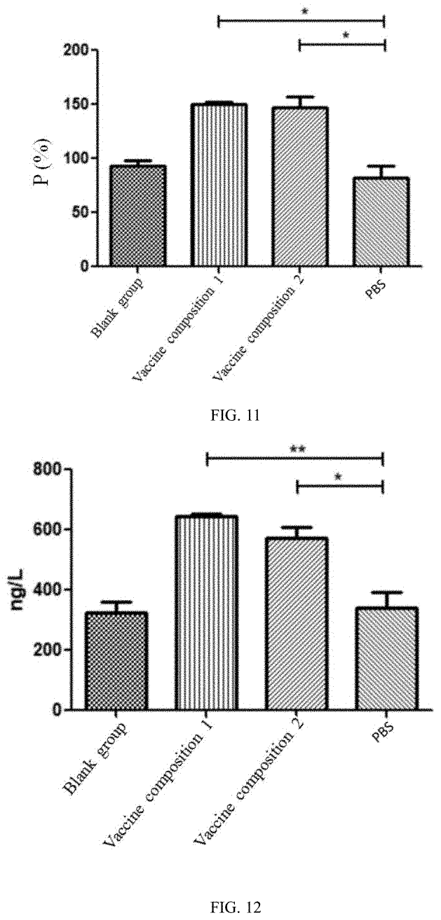

As shown in FIG. 11, 42 days after the first immunization, the relative cell proliferation rates of vaccine composition 1 and vaccine composition 2 in the experimental group are significantly increased as comparing to PBS group (proliferation rate of 82.51%) and blank group (proliferation rate of 92.78%), and compared with PBS group, the percentages of vaccine composition 1 and vaccine composition 2 are increased by 67.24% and 64.38% respectively.

3.4 Detection of Cytokines in Mouse

The supernatant collected in 3.3 is used to detect cytokines including lymphocyte IFN-γ and lymphocyte IL-4, etc.

The mouse interferon-γ (IFN-γ) ELISA kit is used to detect the IFN-γ concentration in the supernatant of spleen lymphocytes with reference to the instruction in the kit.

The mouse interleukin-4 (IL-4) ELISA kit is used to detect the concentration of IL-4 in spleen lymphocyte supernatant with reference to the instruction in the kit.

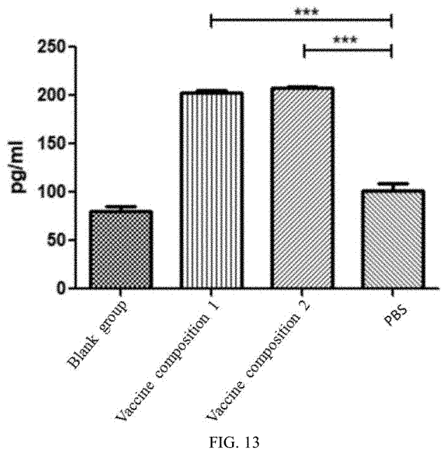

FIG. 12 illustrates the results of the IFN-γ assay. 42 days after the first immunization, the average values of IFN-γ in the vaccine combination 1 and vaccine combination 2 immunized groups are 645.27 ng/L and 572.84 ng/L respectively, both of which are significantly higher compared with the PBS group (342.59 ng/L), and most significant difference is found in the vaccine combination 1 immunized group, which is 302.68 ng/L higher than the value of PBS group.

FIG. 13 shows the results of IL-4 assay. 42 days after the first immunization, the average levels of IL-4 in vaccine composition 1 and vaccine composition 2 immunized groups are 203.3 pg/mL and 207.42 pg/mL respectively, which are significantly higher than those in PBS group (101.82 pg/mL), and the difference between vaccine composition 1 and vaccine composition 2 immunized groups is rather significant, for the values are 101.49 pg/ml and 105.61 higher than that in PBS group respectively.

By constructing the eukaryotic expression plasmid, the disclosure can efficiently express the recombinant protein in mammalian cell lines including 293 cell lines, and the vaccine composition prepared by using the recombinant protein can generate antibodies of high-level after immunizing mice. The results of serum neutralization experiment show that the vaccine composition of the disclosure can effectively stimulate the generation of neutralizing antibodies and hence play a good protective role. The results of detecting spleen lymphocyte subtype and ratio of cytokines show that the vaccine composition of the disclosure can improve the immune function and cytokine expression level of peripheral blood T lymphocytes.

The results of lymphocyte proliferation test show that the vaccine composition of the disclosure can stimulate T cells to transform into immunological memory cells, so when immunological memory cells encounter the same antigen again, a more rapid and effective immune response can be generated.

The embodiments of the disclosure are explained and described in detail with reference to the drawings in the specification, but those skilled in the art should understand that the above embodiments are only preferred embodiments of the disclosure, and the detailed description is only to help readers better understand the spirit of the disclosure, but not to limit the scope of protection of the disclosure; on the contrary, any improvement or modification based on the spirit of the disclosure should fall within the scope of protection of the disclosure.

What is claimed is:

1. A recombinant protein, wherein the recombinant protein is a fusion protein formed of a truncated fragment of spike (S) protein from porcine epidemic diarrhea virus (PEDV) and a Fc fragment of porcine immunoglobulin G (IgG) connected in series.

2. The recombinant protein according to claim 1, wherein the truncated fragment of the S protein comprises:
a segment with an amino acid sequence as shown in SEQ ID NO:1 or an amino acid sequence having the same function as and formed by substituting, deleting and/or adding with one or more amino acid residues into the amino acid sequence as shown in SEQ ID NO:1,
a segment with an amino acid sequence as shown in SEQ ID NO:2 or an amino acid sequence having the same function as and formed by substituting, deleting and/or adding with one or more amino acid residues into the amino acid sequence as shown in SEQ ID NO:2, and
a segment with an amino acid sequence as shown in SEQ ID NO:3 or an amino acid sequence having the same function as and formed by substituting, deleting and/or adding with one or more amino acid residues into the amino acid sequence as shown in SEQ ID NO:3.

3. The recombinant protein according to claim 1, wherein the recombinant protein comprises an amino acid sequence as shown in SEQ ID NO:5 or an amino acid sequence having the same function as and formed by substituting, deleting and/or adding with one or more amino acid residues into the amino acid sequence as shown in SEQ ID NO:5.

4. The recombinant protein according to claim 1, wherein the recombinant protein is a recombinant protein obtained by exogenously expressing a recombinant gene with a deoxyribonucleic acid (DNA) sequence as shown in SEQ ID NO:6 by using an eukaryotic expression system.

5. The recombinant protein according to claim 1, wherein the recombinant protein is a recombinant protein obtained by exogenously expressing a recombinant gene with a DNA sequence as shown in SEQ ID NO:6 by using a mammalian cell expression system.

6. An application method of the recombinant protein according to claim 1,
wherein the recombinant protein is applied in preparing a PEDV vaccine.

7. A vaccine composition of PEDV, wherein the vaccine composition comprises the recombinant protein according to claim 1 and a pharmaceutically acceptable adjuvant.

8. The vaccine composition of PEDV according to claim 7, wherein the adjuvant is one selected from the group consisting of Freund's adjuvant and a layered double hydroxide (LDH) adjuvant.

9. The vaccine composition of PEDV according to claim 8, wherein the vaccine composition is prepared by mixing the recombinant protein and the LDH adjuvant in a mass ratio of 1:4 and emulsifying.

10. The vaccine composition of PEDV according to claim 8, wherein the vaccine composition is prepared by mixing the recombinant protein and Freund's adjuvant in a volume ratio of 1:1 and emulsifying.


SEQUENCE LISTING (7 sequences)

SEQ ID NO: 1 - protein, 215 aa, Porcine epidemic diarrhea virus
LPQDVTRCTANTNFRRFFSKFNVQAPAVVVLGGYLPIGENQGVNSTWYCAGQHPTASGVHGIFLSHIRGGHGFEIGISQEPFDPSGYQLYLHKATNGNTNATARLRICQFPSIKTLGPAANNDVTTGRNCLFNKAIPAHMSEHSVVGITWDNDRVTVFSDKIYYFYFKNDWSRVATKCYNSGGCAMQYVYEPTYYILNVTSAGEDGISYQPCTAN

SEQ ID NO: 2 - protein, 140 aa, Porcine epidemic diarrhea virus
TSFVTLPSFNDHSFVNITVSAAFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTPKPLEGV

SEQ ID NO: 3 - protein, 31 aa, Porcine epidemic diarrhea virus
CTEPVLVYSNIGVCKSGSIGYVRSQSGQVKI

SEQ ID NO: 4 - protein, 224 aa, Pig
PCPICPGCEVAGPSVFIFPPKPKDTLMISQTPEVTCVVVDVSKEHAEVQFSWYVDGVEVHTAETRPKEEQFNSTYRVVSVLPIQHQDWLKGKEFKCKVNNVDLPAPITRTISKAIGQSREPQVYTLPPPAEELSRSKVTVTCLVIGFYPPDIHVEWKSNGQPEPEGNYRTTPPQQDVDGTFFLYSKLAVDKARWDHGETFECAVMHEALHNHYTQKSISKTQGK

SEQ ID NO: 5 - protein, 676 aa, Artificial Sequence
  amino acid sequence in recombinant protein
MYRMQLLSCIALILALVTNSLPQDVTRCTANTNFRRFFSKFNVQAPAVVVLGGYLPIGENQGVNSTWYCAGQHPTASGVHGIFLSHIRGGHGFEIGISQEPFDPSGYQLYLHKATNGNTNATARLRICQFPSIKTLGPAANNDVTTGRNCLFNKAIPAHMSEHSVVGITWDNDRVTVFSDKIYYFYFKNDWSRVATKCYNSGGCAMQYVYEPTYYILNVTSAGEDGISYQPCTANGGGGSGGGGSGGGGSGGGGSTSFVTLPSFNDHSFVNITVSAAFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTPKPLEGVGGGGSGGGGSGGGGSGGGGSCTEPVLVYSNIGVCKSGSIGYVRSQSGQVKIPCPICPGCEVAGPSVFIFPPKPKDTLMISQTPEVTCVVVDVSKEHAEVQFSWYVDGVEVHTAETRPKEEQFNSTYRVVSVLPIQHQDWLKGKEFKCKVNNVDLPAPITRTISKAIGQSREPQVYTLPPPAEELSRSKVTVTCLVIGFYPPDIHVEWKSNGQPEPEGNYRTTPPQQDVDGTFFLYSKLAVDKARWDHGETFECAVMHEALHNHYTQKSISKTQGKHHHHHH

SEQ ID NO: 6 - DNA, 2031 bp, Artificial Sequence
  S1-Fc
atgtacaggatgcagctgctgtcctgtatcgccctgatcctggccctggtgaccaattctctgccacaggacgtgaccaggtgcacagcaaacaccaatttccggagattcttttctaagtttaacgtgcaggcaccagcagtggtggtgctgggaggatacctgccaatcggcgagaatcagggcgtgaactccacatggtattgtgcaggacagcaccctaccgcaagcggagtgcacggcatcttcctgtcccacatcaggggcggccacggcttcgagatcggcatctctcaggagccatttgaccccagcggctaccagctgtatctgcacaaggccacaaacggcaatacaaacgccaccgccaggctgcgcatctgtcagttccctagcatcaagaccctgggcccagccgccaacaatgatgtgaccacaggccgcaattgcctgtttaacaaggccatcccagcccacatgagcgagcactccgtggtgggcatcacatgggacaatgatcgggtgaccgtgttctccgacaagatctactatttctactttaagaacgattggagcagagtggccacaaagtgttataactccggcggctgcgccatgcagtacgtgtatgagccaacatactatatcctgaatgtgacctccgccggcgaggacggcatctcttaccagccctgcaccgccaatggcggcggcggctctggaggaggaggcagcggcggaggaggctccggcggcggcggctctacaagcttcgtgaccctgccctctttcaatgatcacagctttgtgaacatcaccgtgagcgccgcctttggcggacactccggagcaaatctgatcgcatctgacaccacaatcaacggcttcagctccttttgcgtggatacacggcagttcaccatctctctgttttacaatgtgacaaacagctacggctacgtgagcaagtctcaggactccaattgccctttcaccctgcagtctgtgaacgattatctgagcttctccaagttttgcgtgagcaccagcctgctggcaagcgcctgcaccatcgacctgttcggctacccagagtttggctctggcgtgaagtttacaagcctgtatttccagtttaccaagggcgagctgatcaccggcacacccaagcctctggagggagtgggaggaggaggctccggaggaggcggctctggcggcggcggcagcggcggcggcggctcctgtaccgagcctgtgctggtgtacagcaatatcggcgtgtgcaagtccggctctatcggctatgtgaggagccagtccggccaggtgaagatcccatgccccatctgtccaggatgcgaggtggcaggacctagcgtgttcatctttccccctaagcctaaggatacactgatgatctcccagacaccagaggtgacctgcgtggtggtggacgtgtctaaggagcacgccgaggtgcagttcagctggtacgtggatggcgtggaggtgcacacagccgagaccaggcccaaggaggagcagtttaactccacctatcgcgtggtgtctgtgctgcctatccagcaccaggactggctgaagggcaaggagttcaagtgtaaggtgaacaatgtggatctgcctgccccaatcacccggacaatcagcaaggccatcggccagtccagagagccacaggtgtacaccctgccaccacctgcagaggagctgtctcggagcaaggtgaccgtgacatgcctggtcatcggcttttatccacccgatatccacgtggagtggaagagcaatggacagccagagcctgagggaaactacaggaccacacctccacagcaggacgtggatggcacattctttctgtattccaagctggccgtggacaaggccagatgggatcacggcgagaccttcgagtgcgccgtgatgcacgaggccctgcacaaccactacacacagaagtccatctctaagacccagggcaagcaccaccaccaccaccactga

SEQ ID NO: 7 - DNA, 420 bp, Artificial Sequence
  COE gene fragment
acaagcttcgtgaccctgccctctttcaatgatcacagctttgtgaacatcaccgtgagcgccgcctttggcggacactccggagcaaatctgatcgcatctgacaccacaatcaacggcttcagctccttttgcgtggatacacggcagttcaccatctctctgttttacaatgtgacaaacagctacggctacgtgagcaagtctcaggactccaattgccctttcaccctgcagtctgtgaacgattatctgagcttctccaagttttgcgtgagcaccagcctgctggcaagcgcctgcaccatcgacctgttcggctacccagagtttggctctggcgtgaagtttacaagcctgtatttccagtttaccaagggcgagctgatcaccggcacacccaagcctctggagggagtg